SEQUENCE LISTING (16 sequences)

SEQ ID NO: 1 - protein, 178 aa, Artificial
  Mutein of hNGAL
QDSTSDLIPAPPLSKVPLQQNFQDNQFHGKWYVVGLAGNEVLREDKDPMKMWATIYELEEDKSYNVTIVMFLAKKCEYLFQTFVPGSQPGEFTLGDIKSSPGRTSGLVRVVSTNYNQHAMVFFKTVWQNREVFWITLYGRTKELTSELKENFIRFSKSLGLPENHIVFPVPIDQCIDG

SEQ ID NO: 2 - protein, 178 aa, Artificial
  Mutein of hNGAL
QDSTSDLIPAPPLSKVPLQQNFQDNQFHGKWYVVGTAGNSILREDKDPQKMWATIYELKEDKSYNVTRVFFEGKKCRYVIETFVPGSQPGEFTLGKIKSAPGGTSILVRVVSTNYNQHAMVFFKVVWQNRELFWITLYGRTKELTSELKENFIRFSKSLGLPENHIVFPVPIDQCIDG

SEQ ID NO: 3 - protein, 178 aa, Artificial
  Mutein of hNGAL
QDSTSDLIPAPPLSKVPLQQNFQDNQFHGKWYVVGVAGNGLLREDKDPLKMHATIYELKEDKSYNVTRVLFVRKKCRYYISTFVPGSQPGEFTLGRIKSEPGRTSFLVRVVSTNYNQHAMVFFKMVWQNREVFWITLYGRTKELTSELKENFIRFSKSLGLPENHIVFPVPIDQCIDG

SEQ ID NO: 4 - protein, 178 aa, Artificial
  Mutein of hNGAL
QDSTSDLIPAPPLSKVPLQQNFQDNQFHGKWYVVGVAGNEMLREDKDPLKMLATIYELKEDKSYNVTRVMFEYKKCVYLIETFVPGSQPGEFTLGTIKSVPGLTSGLVRVVSTNYNQHAMVFFKRVWQNREVFWITLYGRTKELTSELKENFIRFSKSLGLPENHIVFPVPIDQCIDG

SEQ ID NO: 5 - protein, 178 aa, Artificial
  Mutein of hNGAL
QDSTSDLIPAPPLSKVPLQQNFQDNQFHGKWYVVGAAGNSLLREDKDPMKMWATIYELKEDKSYNVTRVNFGGKKCSYLIETFVPGSQPGEFTLGSIKSRPGATSVLVRVVSTNYNQHAMVFFKLVTQNREVFWITLYGRTKELTSELKENFIRFSKSLGLPENHIVFPVPIDQCIDG

SEQ ID NO: 6 - protein, 178 aa, Artificial
  Mutein of hNGAL
QDSTSDLIPAPPLSKVPLQQNFQDNQFHGKWYVVGLAGNEILREDKDPLKMWATIYELKEDKSYNVTRVQFGEKKCGYGIETFVPGSQPGEFTLGSIKSVPGGTSRLVRVVSTNYNQHAMVFFKFVWQNREVFWITLYGRTKELTSELKENFIRFSKSLGLPENHIVFPVPIDQCIDG

SEQ ID NO: 7 - protein, 178 aa, Artificial
  Mutein of hNGAL
QDSTSDLIPAPPLSKVPLQQNFQDNQFHGKWYVVGLAGNRVLREDKDPQKMFATIYELKEDKSYNVTGVDFRTKKCLYSIGTFVPGSQPGEFTLGVIKSQPGWTSYLVRVVSTNYNQHAMVFFKTVWQNREVFWITLYGRTKELTSELKENFIRFSKSLGLPENHIVFPVPIDQCIDG

SEQ ID NO: 8 - protein, 178 aa, Artificial
  Mutein of hNGAL
QDSTSDLIPAPPLSKVPLQQNFQDNQFHGKWYVVGLAGNEVLREDKDPMKMWATIYELKEDKSYNVTIVMPLAEKCEYLFQTFVPGSQPGEFTLGGIKSGPGRTSGLVRVVSTNYNQHAMVFFKVVWQNREVFWVTLYGRTKELTSELKENFIRFSKSLGLPENHIVFPVPIDQCIDG

SEQ ID NO: 9 - protein, 178 aa, Artificial
  Mutein of hNGAL
QDSTSDLIPAPPLSKVPLQQNFQDNQFHGKWYVVGLAGNEVLREDKDPMKMWATIYELKEDKSYNVTIVMSLAKKCEYLFQTFVPGSQPGEFTLGDIKSSPGRTSGLVRVVSTNYNQHAMVFFKVVWQNREVFWITLYGRTKELTSGLKENFIRFSKSLGLPENHIVFPVPIDQCIDG

SEQ ID NO: 10 - protein, 179 aa, Artificial
  Mutein of hNGAL
QDSTSDLIPAPPLSKVPLQQNFQDNQFHGKWYVVGLAGNEILREDKDPMKMWATIYELKEDRSYNVTIVMFLAKKCEYLFQTFVPGSQPGEFTLGDIKSSPGRTSGLVRVVSTNYNQHAMVFFKVVWQNREVFWITLYGRTKELTPELKENFIRFSKSLGLPENHIVFPVPIDQCIDGF

SEQ ID NO: 11 - protein, 178 aa, Artificial
  Mutein of hNGAL
QDSTSDLIPAPPLSKVPLQQNFQDNQFHGKWYVVGLAGNEILREDKDPMKMWATIYELKEDKSYNVTIVMFLAKKCEYLFQTFVPGSQPGEFTLGDIKSSPGRTSGLVRVVSTNYNQHAMVFFKVVWQNREVFWITLYGRTKELTSGLKENFIRFSKSLGLPENHIVFPVPIDQCIDG

SEQ ID NO: 12 - protein, 178 aa, Artificial
  Mutein of hNGAL
QDSTSDLIPAPPLSKVPLQQNFQDNQFHGKWYVVGLAGNEVLREDKDPMKMWATIYELKEDKSYNVTIVMFLAEECEYLFQTFVPGSQPGEFTLGDIKSSPGRTSGLVRVVSTNYNQHAMVFFKVVWQNREVFWITLYGRTKELTSELKKNFIRFSKSLGLPENHIVFPVPIDQCIDG

SEQ ID NO: 13 - protein, 178 aa, Artificial
  Mutein of hNGAL
QDSTSDLIPAPPLSKVPLQQNFQDNQFHGKWYVVGLAGNEVLREDKDPMKMWATIYELKEDKSYNVTIVMPLAEKCEYLFQTFVPGCQPGEFTLGGIKSGPGRTSGLVRVVSTNYNQHAMVFFKVVWQNREVFWVTLYGRTKELTSELKENFIRFSKSLGLPENHIVFPVPIDQCIDG

SEQ ID NO: 14 - protein, 178 aa, Artificial
  Mutein of hNGAL
QDSTSDLIPAPPLSKVPLQQNFQDNQFHGKWYVVGLAGNEVLREDKDPMKMWATIYELEEDKSYNVTIVMFLAKKCEYLFQTFVPGCQPGEFTLGDIKSSPGRTSGLVRVVSTNYNQHAMVFFKTVWQNREVFWITLYGRTKELTSELKENFIRFSKSLGLPENHIVFPVPIDQCIDG

SEQ ID NO: 15 - protein, 178 aa, human
  wild type hNGAL
QDSTSDLIPAPPLSKVPLQQNFQDNQFHGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 16 - protein, 25 aa, human
  Hepcidin-25
DTHFPICIFCCGCCHRSKCGMCCKT

(19) United States
(12) Patent Application Publication
(10) Pub. No.: US 2015/0369821 A1
(43) Pub. Date: Dec. 24, 2015

(54) NOVEL LIPOCALIN-MUTEIN ASSAYS FOR MEASURING HEPCIDIN CONCENTRATION

(71) Applicant: PIERIS AG, Freising-Weihenstephan (DE)

(72) Inventors: Stefan TRENTMANN, Allershausen (DE); Rachida SIHAM BEL AIBA, Munich (DE); Andrea ALLERSDORFER, Geisenhausen (DE); Nicole ANDERSEN, Freising (DE); Andreas HOHLBAUM, Paunzhausen (DE)

(73) Assignee: PIERIS AG, Freising-Weihenstephan (DE)

(21) Appl. No.: 14/765,933

(22) PCT Filed: Feb. 5, 2014
(86) PCT No.: PCT/EP2014/052228

§ 371 Date: Aug. 5, 2015

Related U.S. Application Data
(60) Provisional application No. 61/761,456, filed on Feb. 6, 2013.

(51) Int. Cl. G01N 33/68

(52) U.S. Cl. CPC G01N 33/6872; G01N 2333/4703

(57) ABSTRACT

The present invention relates to lipocalin-mutein assays for measuring hepcidin concentration as well as methods preparing and utilizing and kits leveraging the lipocalin-mutein assays.

### I. BACKGROUND

Hepcidin is a small cysteine-rich peptide predominantly produced in the liver. This peptide regulates the absorption of iron in the intestine and inhibits release of iron from macrophages (Nicolas et al., Proc Natl Acad Sci USA 2001; 98, 8780-8785). This peptide plays a pivotal role in iron metabolism (Nicolas et al., Proc Natl Acad Sci USA 2002; 99, 4596-4601), and is a central regulator of iron homeostasis (Ahmad et al., Blood Cells Mol Dis. 2002; 29, 361-366), therefore, hepcidin could become a useful biomarker for the diagnosis and monitoring of e.g. iron disorders (Kroot et al., Hepcidin in human iron disorders: diagnostic implications; Clin Chem. 2011; 57:1650-1669).

In recent years, numerous methods using mass spectrometry (MS) as the reliable ways to quantify hepcidin (such as matrix assisted laser desorption/ionization time-of-flight mass spectrometry (MALDI-TOF MS), surface enhanced laser desorption/ionization time-of-flight mass spectrometry (SELDI-TOF MS) and liquid chromatography tandem-mass spectrometry techniques (LC-MS MS)) have been published. Published MS methods offer high sensitivity, and, with the use of a stable isotope internal standard, high accuracy, but are generally restricted by low throughput workflows (see e.g. Bansal et al., Anal Biochem. 2009; 384:245-253.). A recently described method involving off-line WCX magnetic bead-based enrichment prior to traditional dried droplet spotting and MALDI-TOF analysis benefits from isotopic resolution and enhanced accuracy compared with SELDI, however, the high throughput capacity of the assay and it's applicability in serum or plasma were not demonstrated (Bansal et al., Rapid Commun Mass Spectrom. 2009; 23:1531-1542). In general, the methodological complexities and restrictions of existing MS methods limit their use in large scale clinical applications, which are often resource/labor-intensive, require more costly and sophisticated instrumentation, demand high sample throughput, and, in certain cases, may be constrained by limited sample volumes.

Thus, although MS methods may promise to be more accurate when compared to immunoassays (e.g. immunochemical (IC) assays), they are less practical for routine clinical use at the present time. On the other hand, while immunoassays have the potential of more widespread use among clinical laboratories, progress in developing conventional immunochemical (IC) hepcidin assays has been hampered by, for example, the difficulty in both generating hepcidin-specific reagents with sufficiently high affinity and identifying the suitable assay formats for such reagents e.g. to ensure the sensitivity of the assay or the accuracy of standard curves generated therefrom. At present, there are considerable differences in hepcidin measurements using IC methods vis-à-vis MS approaches. The two international rounds “Round Robin-1 and Round Robin-2” towards the harmonization of hepcidin measurements have highlighted these differences in hepcidin measurements (Swinkels et al., Results of the first international round robin for the quantification of urinary and plasma hepcidin assays: need for standardization. Haematologica. 2009; 94:1748-1752; Swinkels et al., Second round robin for plasma hepcidin methods: first steps toward harmonization. Am J Hematol. 2012; 87:977-983), suggesting great care needs to be exercised in both correlating hepcidin concentrations determined using different methods and relying on IC methods readout, given the high potential for deviation from MS methods readouts.

In this regard, the present application provides an alternative approach for the quantitative measurement of hepcidin concentration in a biological sample or in a subject, which approach is capable of determining hepcidin concentrations in the same range as expected from a benchmark MS approach (as essentially described in Murphy A T et al., Blood. 2007; 110:1048-1054) and with a low limit of detection, and thus can measure hepcidin concentrations as accurate as the MS approach but is more convenient for high-throughput analyses of e.g. serum samples at lower cost compared with MS-based methods when widely used in clinical settings.

### II. INTRODUCTION

In one aspect, the current application features a lipocalin-mutein assay that can be useful for quantitatively measuring hepcidin concentrations; and thereby, in some embodiments, identifying an altered, e.g. increased or reduced, level of hepcidin concentration. In another aspect, the present disclosure relates to a lipocalin-mutein assay that can be useful for diagnosing a disease or disorder characterized by a non-physiological concentration of hepcidin. Uses of a lipocalin-mutein assay of the disclosure may, in some embodiments, involve assessing the hepcidin concentration in a biological sample obtained from a subject.

The lipocalin-mutein assays of the disclosure are set up using competition formats, based on the binding of one or more lipocalin muteins, or fragments or variants, specifically to hepcidin, as provided in detail below. The current disclosure opens a broad range of perspectives in that a variety of methods and kits leveraging one or more lipocalin-mutein assays of the disclosure can be widely applicable for different diagnostic purposes.

### III. DEFINITIONS

The term “hepcidin” refers to the protein also termed liver-expressed antimicrobial peptide 1 or putative liver tumor regressor, the human form of which has the UniProtKB/Swiss-Prot accession number P81172. On a general basis, the term “hepcidin” refers to any form of the hepcidin protein known to be present in vertebrate species, including in mammals, but preferably, in primates (e.g. Cynomolgous monkeys or humans) and includes, but is not limited to any mature, bioactive form of the hepcidin protein expressed in a vertebrate such as a mammal.

The term “human hepcidin” refers to any form of the hepcidin protein present in humans. The human unprocessed protein has a length of 84 amino acids and is encoded by the gene “HAMP,” also known as “HEPC” or “LEAP1.” It is cleaved into two chains, which are herein also included in the term “human hepcidin.” These two chains are of amino acids 60-84, which is Hepcidin-25 (Hepc25), and of amino acids 65-84, which is Hepcidin-20 (Hepc20), respectively. Hepcidin-25 is arranged in the form of a bent hairpin, stabilized by four disulfide bonds. Natural variants of human hepcidin, also included in the term “human hepcidin”, have, for example, the amino acid replacement 59 R→G (VAR_{—}0425129); the amino acid replacement 70 C→R (VAR_{—}042513); the amino acid replacement 71 G→D (VAR_{—}026648) and/or the amino acid replacement 78 C→Y (VAR_{—}042514). A further natural variant is Hepcidin-22, another N-terminally truncated isoform (besides Hepcidin-20) of Hepcidin-25. The expression “Hepcidin-25” refers to the mature form of human hepcidin with the amino acid sequence as depicted in SEQ ID NO: 16. A hepcidin molecule may only be present in a biological sample, without having any measurable physiological relevance. For example, Hepcidin-22 so far has only been detected in urine and so far is assumed to merely be a urinary degradation product of Hepcidin-25 (reviewed in Kemna et al., Haematologica. 2008 January; 93:(1)90-97). In some embodiments, one or more lipocalin muteins of the disclosure are able to bind each given form of human hepcidin including proteolytic fragments thereof, regardless of whether the respective hepcidin molecule displays biological/physiological activity. A lipocalin mutein of the disclosure may also bind physiological active species such as the mature, bioactive Hepcidin-25.

The term “subject” refers to a vertebrate animal, including a mammal, and in particular a human, in which case the term “patient” can also be used. In some embodiments, the subject may have a disorder that would benefit from an increase in iron in serum, reticulocyte count, red blood cell count, hemoglobin, and/or hematocrit.

The term “biological sample” refers to any fluid, tissue or other material derived from the body of a normal or diseased subject, such as blood, serum, plasma, lymph, urine, saliva, tears, cerebrospinal fluid, milk, amniotic fluid, bile, ascites fluid, pus and the like. Also included within the meaning of this term are an extract organ and a culture fluid in which any cells or tissue preparation from the subject that has been incubated.

### IV. BRIEF DESCRIPTION OF FIGURES

FIG. 1—an exemplary standard curve of a lipocalin-mutein assay indicating a linear range of 1-185 ng/mL—shows in an electrochemiluminescense-based assay set up according to Example 3, a constant concentration of Sulfo-Tag-labeled control hepcidins competed for binding to lipocalin muteins of SEQ ID NO: 10 with various known concentrations of unlabeled hepcidins (non-control hepcidins) to generate a standard curve, which showed a linear range from 1 ng/mL up to 185 ng/mL and wherein generated signals were plotted versus said various concentrations.

FIG. 2—an exemplary standard curve generated by a lipocalin-mutein assay indicating a linear range of 2-185 ng/mL—depicts in an enzyme-linked fluorescence-based assay set up according to Example 4, a constant concentration of C-terminal biotinylated control hepcidins (hepcidin-C-Bios) competed for binding to lipocalin muteins of SEQ ID NO: 10 with various known concentrations of unlabeled hepcidins (non-control hepcidins) to generate a standard curve, which showed a linear range from 2 ng/mL up to 185 ng/mL, wherein the hepcidin-C-Bios were detected via Extravidin-HRP and generated signals were plotted versus said various concentrations.

FIG. 3—an exemplary standard curve generated by a lipocalin-mutein assay indicating a linear range of 0.8-555 ng/mL—illustrates in an enzyme-linked absorption-based assay set up according to Example 10, a constant concentration of C-terminal biotinylated control hepcidins (hepcidin-C-Bios) competed for binding to lipocalin muteins of SEQ ID NO: 10 with various known concentrations of unlabeled hepcidins (non-control hepcidin) to generate a standard curve, which showed a linear range from 0.8 ng/mL up to 555 ng/mL, wherein the hepcidin-C-Bios was detected via Extravidin-HRP and generated signals were plotted versus said various concentrations.

### V. DETAILED DESCRIPTION OF THE DISCLOSURE

For quantifying the amount of hepcidins in a biological sample of a subject, the present disclosure provides one or more lipocalin-mutein assays based on the binding of one or more lipocalin muteins, or fragments or variants thereof, specifically to hepcidin as well as ways to analyze data generated therefrom.

In this regard, one or more lipocalin-mutein assays of the disclosure may contain a tracer molecule that can be can be captured on a phase by a capture reagent. In addition, such tracer molecule may be detected and/or quantified via a label, for example, through a suitable device or machine as known in the art. In this regard, the tracer molecule can either be detected and/or quantified directly when the tracer molecule is labeled, or be detected and/or quantified indirectly via another labeled molecule that can directly or indirectly bind to the tracer molecule. As used in this context, the term “phase” means a surface where the tracer molecule can be bound to.

In one embodiment, the signal, such as electronic signal, radioactivity, luminescence, color or the like, developed by the label is a direct measurement of the amount of captured tracer molecules. In another embodiment, the amount of captured tracer molecules may be measured indirectly. In some embodiments, a label of the disclosure, when used in a lipocalin-mutein assay as disclosed herein, may be read and/or measured, using a method appropriate to the label as known in the art.

In one aspect of the current application, the tracer molecule may be a control hepcidin including fragment or variant thereof while the capture reagent may be a lipocalin mutein including fragment or variant thereof as disclosed herein. In yet another aspect of the current application, however, the tracer molecule may be a lipocalin mutein including fragment or variant thereof as disclosed herein while the capture reagent may be a control hepcidin including fragment or variant thereof. To maximize the sensitivity of the lipocalin-mutein assays of the disclosure over the range of interest and to ensure the accuracy of standard curves generated therefrom, in some preferred embodiments, the concentration of the tracer molecule is critical. Therefore, its concentration can range between about 0.1 nM-3 nM in such assays.

In some further embodiments, the tracer molecule is at the concentration of about 1 nM-3 nM in a lipocalin-mutein assay of the disclosure. In some still further embodiments, the tracer molecule is at the concentration of about 0.4 nM, about 0.5 nM, about 0.6 nM or about 0.7 nM in a lipocalin-mutein assay of the disclosure.

In some embodiments, one or more lipocalin-mutein assays as disclosed herein may include one or more control hepcidins that compete with non-control hepcidins (e.g. hepcidins in a biological sample) for binding to one or more lipocalin muteins or fragments or variants thereof as disclosed herein. The term “control hepcidin”, when used as disclosed herein, includes, but is not limited to, synthetic hepcidin, isolated and/or purified hepcidin from a subject, and recombinant hepcidin.

In this regard, a fragment of a control hepcidin refers to proteins or peptides derived from a full-length mature hepcidin as well as its natural variants but are N-terminally and/or C-terminally shortened, i.e. lacking at least one of the N-terminal and/or C-terminal amino acids. Such fragments include preferably at least 5 or more (e.g. 9) consecutive amino acids of the primary sequence of mature human hepcidin (Hepcidin-25) as well as its natural variants (e.g. Hepcidin-22) and are usually detectable in an immunoassay of mature human hepcidin. Such fragments of hepcidin comprise small peptides that mimic the action of hepcidin, such as mini-hepcidin peptides (Preza G C, Ruchala P, Pinon R, et al., Analysis of the hepcidin-ferroportin interface yields minihepcidins, small peptides for the treatment of iron overload. J Clin Invest. In press). In addition, a variant of a control hepcidin refers to derivatives of any form of the hepcidin protein present in nature (e.g. human hepcidin defined above) that comprise modifications of the amino acid sequence, for example by substitution, deletion, insertion or chemical modification. Preferably, such modifications do not reduce the functionality of the hepcidin protein.

In relation to such lipocalin-mutein assays, a control hepcidin or fragment or variant thereof may be conjugated to a moiety and thereby can be captured by a binding agent. In addition, in some embodiments, a control hepcidin or fragment or variant thereof, when included in a lipocalin-mutein assay of the disclosed, may be directly or indirectly labelled. In contrast, non-control hepcidins as used in the present disclosed refer to those hepcidins whose concentration (e.g. in a biological sample) can be measured or determined using a lipocalin-mutein assay of the disclosure. In some preferred embodiments, such non-control hepcidins need not be labelled or conjugated for the purpose of applying a lipocalin-mutein assay of the disclosure.

In some further embodiments, the lipocalin-mutein assays of the disclosure may further comprise one or more binding agents, wherein a control hepcidin or fragment or variant thereof is conjugated to a moiety and thereby can be captured by such binding agents. For example, in a particular embodiment, a control hepcidin or fragment or variant thereof may be conjugated to a biotin that allows binding of e.g. multiple streptavidin, avidin or Neutravidin to conjugated control hepcidin.

Moreover, in some further embodiments, the mean value of the concentration of non-control hepcidins in a biological sample, as measured by one or more lipocalin-mutein assays as disclosed herein, is within the same range of the mean value of the concentration of non-control hepcidins in a corresponding sample as measured by a mass spectrometry (MS) assay. In this regard, a corresponding sample is the same type of sample as the biological sample mentioned earlier and obtained from the same subject; namely, if the biological sample is a serum sample obtained from a subject, the corresponding sample should also be a serum sample taken from the same subject. In addition, when used in this context, the “mean value” is defined as the arithmetic mean of two or more values when the amount of non-control hepcidins in a biological sample is measured at n time points (either by a lipocalin-mutein assay of the disclosure or by a MS assay), computed by first adding together the numbers as measured at each time point and then dividing the total number by n, as representatively illustrated in Example 6. In some further embodiments, the MS assay is essentially described in Murphy A T et al., Blood. 2007; 110:1048-1054 as referred in Example 6. In addition, when used herein, the “same range” means that the difference between two values is less than 50% of the higher one of the two values. In some further embodiments, the “same range” means that the difference between two values is less than 30% of the higher one of the two values. In some still preferred embodiments, the “same range” means that the difference between two values is less than 10% of the higher one of the two values.

The term “lipocalin-mutein assay”, when used as disclosed herein, in principle is similar to the immunoassay known in the art except that one or more lipocalin muteins instead of one or more immunoglobulins are used in the assay. Such immunoassay known in the art includes, but is not limited to, immunochemical (IC) assays such as radioimmunoassay (RIA), fluoroluminescence assay (FLA), chemiluminescence assay (CA), and enzyme-linked immunosorbant assay (ELISA). ELISA methods are described, for example, in WO01/36972. In addition, the immunoassay also includes electrochemiluminescent assays (ECLA). As used herein, “electrochemiluminescence assay” or “ECLA” is an electrochemical assay in which an electrode electrochemically initiates luminescence of a chemical label. Light emitted by the label is measured by a photodetector and indicates the presence or quantity of bound hepcidin. ECLA methods are described, for example, in U.S. Pat. Nos. 5,543,112; 5,935,779 and 6,316,607. In some embodiments, signal modulation can be maximized for different hepcidin concentrations for precise and sensitive measurements.

In this regard, the assays of the disclosure are not strictly “immuno” assays, though the names of some of those assays might carry the original “immuno” because of the common use and history of development of such.

The term “label”, when used as disclosed herein, is a substance that is capable of developing a detectable signal, for example, can convert a colorless substrate into a colored product; and depending on the type of the assay utilized, the term “label” of the disclosure includes, but is not limited to, a chemical moiety, a radioactive label, a photoluminescent label, a fluorescent label, a chemiluminescent label, an enzyme, an electrochemiluminescent label and the like. In a particular embodiment, the label is a Sulfo-Tag. In another particular embodiment, the label is a HRP.

In some embodiments, one or more lipocalin-mutein assays of the disclosure may further comprise a blocking agent as described below.

In one embodiment, the present disclosure also concerns a method of preparing a lipocalin-mutein assay of the disclosure, which method may comprise immobilizing one or more lipocalin muteins or fragments or variants thereof on a phase. In some further embodiments, the method of preparing a lipocalin-mutein assay of the disclosure may further comprise providing one or more control hepcidins or fragments or variants thereof. In some further preferred embodiments, the control hepcidins or fragments or variants thereof are provided at the concentration range of 0.1 nM-3 nM.

In yet another embodiment, the present disclosure features a method of preparing a lipocalin-mutein assay of the disclosure, which method may comprise immobilizing one or more binding agents on a phase. In some further embodiments, the method of preparing a lipocalin-mutein assay of the disclosure may further comprise providing one or more control hepcidins or fragments or variants thereof, wherein the control hepcidins or fragments or variants thereof may be conjugated to a moiety and thereby can be captured by such binding agents. In some still further embodiments, the method may further comprise providing one or more lipocalin muteins or fragments or variants thereof. In a particular embodiment, the binding agents may be biotin-binding agents e.g. NeutrAvidins, while the control hepcidins or fragments or variants thereof may be conjugated with biotin and thereby is biotinylated. In some further preferred embodiments, the lipocalin muteins or fragments or variants thereof are provided at the concentration range of 0.1 nM-3 nM.

In some preferred embodiments, a method of preparing a lipocalin-mutein assay of the disclosure may further comprise adding a blocking agent as described below.

When applied in one or more lipocalin-mutein assays of the disclosure, a tracer molecule as disclosed herein, in one aspect, may be labeled directly, namely directly linked or fused to a label. In another aspect, a tracer molecule herein may be labeled indirectly, for example, bound with an additional binding agent that may be either directly linked or fused to a label or may be bound with a labeled further binding agent.

In some embodiments where the tracer molecule is a lipocalin mutein including fragment or variant thereof, the lipocalin muteins or fragments or variants thereof may be directly labelled, namely directly linked with or fused to a label. In some further embodiments, the lipocalin muteins or fragments or variants thereof may be indirectly labeled. In this regard, in some still further embodiments, the lipocalin-mutein assays may further comprise one or more additional binding agents, for example, immunoglobulins such as antibodies, to capture lipocalin muteins or fragments or variants thereof. In one case, the additional binding agents may be directly labeled, namely directly linked with or fused to a label. Alternatively, the additional binding agents may be in turn captured by one or more labeled further binding agents, for example, labeled immunoglobulins.

In some other embodiments where the tracer molecule is control hepcidin, control hepcidin may be directly labeled, namely directly linked with or fused to a label. In some further embodiments, control hepcidin may be indirectly labeled. In this regard, control hepcidin may be conjugated to a moiety and thereby can be captured by a labeled additional binding agent. For example, the labeled additional binding agent may be a biotin-binding agent (e.g. streptavidin) that is linked with or fused to a label, while control hepcidin may be conjugated with biotin and thereby is biotinylated.

Linking a label of the disclosure with a tracer molecule (e.g. control hepcidin or lipocalin mutein including fragment or variant thereof, as the case may be), an additional binding agent (e.g. biotin-binding agent such as streptavidin, avidin or Neutravidin; and immunoglobulin such as antibody, as the case may be) or a further binding agent (e.g. immunoglobulin such as antibody) is a standard manipulative procedure in immunoassay techniques, which procedure is transferable for lipocalin-mutein assays of the disclosure.

In some embodiments, a lipocalin-mutein assay of the disclosure is a lipocalin-mutein chemical assay, wherein the tracer molecule is labeled with a label selected from the group consisting of a chemical moiety, a radioactive label, a photoluminescent label, a fluorescent label, a chemiluminescent label and an enzyme.

In some other embodiments, a lipocalin-mutein assay of the disclosure is an electrochemiluminescence assay (ECLA), wherein the tracer molecule is labeled with an electrochemiluminescent label.

In some embodiments, each one of a tracer molecule, an additional binding agent and a further binding agent, as disclosed herein, may be tagged with the label and incubated at room temperature. The incubation time may be from about 0.25 to 3 hours. The pH of the incubation buffer is chosen to maintain a significant level of specific binding of a molecule referred above to its target of interest (e.g. one or more lipocalin muteins, including fragments or variants thereof, to hepcidin). In an embodiment, the pH of the incubation buffer is about 6-9.5, more preferably about 6-7. Various buffers can be employed to achieve and maintain the desired pH during this step, including borate, phosphate, carbonate, Tris-HCl or Tns-phosphate, acetate, barbital and the like. However, the particular buffer employed is usually not critical in individual assays, while in some particular embodiments, one buffer may be preferred over another. The pH and/or temperature of the system may also be varied.

In some embodiments, a lipocalin-mutein assay of the disclosure can be a solid phase assay or a liquid phase assay, wherein least one molecule under analysis is bound to a surface while some other reactants being free in solution. In some further embodiments, one or more lipocalin muteins including fragments or variants thereof or one or more binding agents (such as biotin binding agents including NeutrAvidin) are immobilized on a solid phase or a liquid phase.

In some still preferred embodiments, the lipocalin-mutein assay is a solid phase assay (e.g. where walls of a microplate or sides of a tube are used as the surface). In this regard, immobilization of one or more lipocalin muteins of the disclosure including fragments or variants thereof or of one or more binding agents (such as biotin binding agents including NeutrAvidin), to a solid phase can be conventionally accomplished by insolubilizing such lipocalin muteins including fragments or variants thereof or such binding agents (e.g. biotin binding agents including NeutrAvidin) either before the assay procedure, as by adsorption to a water-insoluble matrix or surface (see, for example, U.S. Pat. No. 3,720,760) or non-covalent or covalent coupling, for example, using glutaraldehyde or carbodiimide cross-linking, with or without prior activation of the support with, for example, nitric acid and a reducing agent e.g. as described in U.S. Pat. No. 3,645,852 or in Rotmans et al., 1983, J. Immunol. Methods, 57:87-98, or after the assay procedure, for example, by immunoprecipitation.

In some embodiments, the solid phase used for immobilization can be any inert support or carrier that is essentially water insoluble and useful in immunoassays, including supports in the form of, for example, surfaces, particles, porous matrices and the like. Examples of commonly used supports include small sheets, Sephadex, polyvinyl chloride, plastic beads, microparticles, assay plates, or test tubes manufactured from polyethylene, polypropylene, polystyrene and the like. Such supports include, but is not limited to multi-well microtiter plates (e.g. with 96 or 384 wells), as well as particulate materials such as filter paper, agarose, cross-linked dextran, and other polysaccharides. Alternatively, reactive water-insoluble matrices such as cyanogen bromide-activated carbohydrates and the reactive substrates (e.g. as described in U.S. Pat. Nos. 3,969,287; 3,691,016; 4,195,128; 4,247,642; 4,229,537 and 4,330,440) may be employed for immobilization. In a particular embodiment, the immobilized lipocalin muteins including fragments or variants thereof or binding agents (such as biotin binding agents including NeutrAvidin) are coated on a microtiter plate. In some still preferred embodiments, the solid phase is a multi-well microtiter plate that can be used to analyze several samples at one time.

In some embodiments, coating the solid phase with lipocalin muteins including fragments or variants thereof or with binding agents (such as biotin binding agents including NeutrAvidin) can be accomplished by a non-covalent or covalent interaction or physical linkage, as desired. Techniques for such attachment include those described in U.S. Pat. No. 4,376,110 and the references cited therein. If covalent attachment of lipocalin muteins including fragments or variants thereof or of binding agents (such as biotin binding agents including NeutrAvidin) to the plate is utilized, the plate or other solid phase can, in some embodiments, be incubated with a cross-linking agent together with lipocalin muteins including fragments or variants thereof or with binding agents (such as biotin binding agents including NeutrAvidin). Commonly used cross-linking agents for attaching lipocalin muteins including fragments or variants thereof or binding agents (such as biotin binding agents including NeutrAvidin) to the solid phase substrate include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates capable of forming cross-links in the presence of light.

If microtiter plates are utilized, the wells in the plate are coated with lipocalin muteins including fragments or variants thereof or with binding agents such as biotin binding agents (for example, diluted in a buffer), preferably, by incubation for a several hours or overnight, at temperatures between 4-37° C. and at a pH of about 6-12. The plates can be stacked and coated in advance of the assay, allowing for an immunoassay to be carried out simultaneously on several samples in a manual, semi-automatic, or automatic fashion, such as by using robotics.

In some embodiments, the coated plates can be treated with a blocking agent that binds non-specifically to, and saturates, the binding sites to prevent unwanted binding of e.g. free ligand other than the molecule of interest to excess binding sites on the wells of the plate. Examples of appropriate blocking agents include, for example, gelatin, bovine serum albumin, egg albumin, casein, and non-fat milk. The blocking treatment typically takes place under conditions of ambient temperatures for about 1-4 hours, preferably about 1 to 3 hours.

In some embodiments, after coating and/or blocking, a wash solution may be used to remove uncaptured molecules from the phase. The wash solution is generally a buffer. The incubation buffers described above are suitable wash solutions. The pH of the wash solution is determined as described above for the incubation buffer. In an embodiment, the pH of the wash solution is about 6-9, more preferably about 6-7. Washes can be done one or more times, preferably, at least three times to reduce the background noise of the assay. The temperature of the wash solution is typically from about 0-40° C., more preferably about 4-30° C. An automated plate washer can be utilized.

Buffers that can be used for dilution, incubation and/or washing include, for example:

(a) phosphate buffered saline (PBS) containing 0.5% BSA, 0.05% TWEEN20™ detergent (P20), 5 mM EDTA, 0.25% Chaps surfactant, 0.2% beta-gamma globulin, and 0.35M NaCl, pH 7.0;
(b) PBS containing 0.5% BSA and 0.05% P20;
(c) PBS containing 0.5% BSA, 0.05% P20, 5 mM EDTA, and 0.35 M NaCl, pH 6.35;
(d) PBS containing 0.5% BSA, 0.05% P20, 5 mM EDTA, 0.2% beta-gamma globulin, and 0.35 M NaCl;
(e) PBS containing 0.5% BSA, 0.05% P20, 5 mM EDTA, 0.25% Chaps, and 0.35 M NaCl; and
(f) PBS containing 0.5% P20.

Furthermore, in some embodiments, the present disclosure relates to one or more methods for quantitatively measuring a biological sample's hepcidin concentration, which methods comprise: (i) contacting a biological sample obtained from a subject with a lipocalin-mutein assay of the disclosure, (ii) and measuring the signal level generated by one or more tracer molecules, captured on the phase, via one or more labels and/or a suitable instrument for signal detection, and (iii) correlating the signal level on a standard curve with the biological sample's hepcidin concentration

In some further embodiments, the methods for quantitatively determining a biological sample's hepcidin concentration further comprise: (iv) contacting various known concentrations of non-control hepcidins with the lipocalin-mutein assay; and (v) measuring the signal levels corresponding to the various concentrations of step (iv) to generate a standard curve, which signal levels are generated by one or more tracer molecules, captured on the phase, via one or more labels and/or a suitable instrument for signal detection. In a particular embodiment, the steps (iv) and (v) is carried out simultaneously with steps (i) and (ii) mentioned above, respectively.

In some other embodiments, however, the methods for quantitatively determining a biological sample's hepcidin concentration may be implemented using a consolidated standard curve that is generated by one or more repetitions of the methods of the disclosure. In this regard, the methods for quantitatively determining a biological sample's hepcidin concentration may also be carried out without steps (iv) and (v) mentioned above.

In a further embodiment, multiple repetitions may be required to identify an absolute linear range for a standard curve. In some circumstances, a further optimization of the lipocalin-mutein assay may be desired.

In addition, in various embodiments, the present disclosure provides methods for identifying an altered, e.g. increased or reduced, level of hepcidin concentration in a subject, which methods comprise: (i) quantitatively measuring a biological sample's hepcidin concentration using a method of the disclosure, wherein the biological sample is obtained from the subject; and (ii) hepcidin concentration measured in step (i) with the prior-measured hepcidin concentration(s) of one or more corresponding sample(s) obtained from the subject. In some further embodiments, the corresponding sample(s)′ hepcidin concentration(s) have been measured using a method of the disclosure. In this regard, a corresponding sample is the same type of sample as the biological sample mentioned earlier and obtained from the same subject; namely, if the biological sample is a serum sample obtained from a subject, the corresponding sample should also be a serum sample taken from the same subject.

In some yet other embodiments, the present disclosure also features methods for diagnosing a disease or disorder characterized by a non-physiological hepcidin concentration in a subject, which methods comprise: (i) quantitatively measuring a biological sample's hepcidin concentration using a method of the disclosure; and (ii) analyzing whether the hepcidin concentration measured in step (i) is non-physiological, wherein the non-physiological concentration of hepcidin is an indicative of the disease or disorder in the subject.

In some further embodiments, the analysis in step (ii) may include comparing the hepcidin concentration measured in step (i) with the hepcidin concentration of a control sample, which is known to possess a normal hepcidin concentration, since it may thus be determined that whether a non-physiological hepcidin concentration is present in the subject. In some other embodiments, the measured hepcidin concentration is so deviating from the normal range of hepcidin concentrations in the kind of samples for such subject, as known in the art (see, for example, age- and sex-specific reference ranges of serum hepcidin concentration provided in Galesloot et al., Serum hepcidin: reference ranges and biochemical correlates in the general population. Blood. 2011; 117:e218-225; however, it should also be noted that the state of art may evolve in the future and provide a renewed standard for the normal range of hepcidin concentrations in the kind of samples for such subject, based on data stratified from larger population using e.g. methods, assays and kits of this disclosure) that It may be thus determined that a non-physiological hepcidin concentration is present in the subject.

In some embodiments, in relation to the methods of the disclosure, one or more biological samples as well as various known concentrations of non-control hepcidins may be diluted as necessary and added to the immobilized phase. The preferred dilution rate is about 5-15%, preferably about 10%, by volume.

In some embodiments, in relation to the methods of the disclosure, one or more biological samples as well as various known concentrations of non-control hepcidins may be incubated with a lipocalin-mutein assay of the disclosure. In this regard, conditions for the incubation may be selected to maximize sensitivity of the assay and to minimize dissociation, e.g. the pH and/or temperature of the system can be varied.

Incubation time depends primarily on the temperature. Preferably, the incubation time may be from about 0.5 to 3 hours. To maintain the sensitivity of a lipocalin mutein assay of the disclosure, incubation times greater than about 10 hours are avoided if possible. If the sample is a biological fluid, incubation times can be lengthened by adding a protease inhibitor to the sample to prevent proteases in the biological fluid from degrading the analyte, hepcidin.

The pH of the incubation buffer is chosen to maintain a significant level of specific binding of a molecule referred above to its target of interest (e.g. one or more lipocalin muteins, including fragments or variants thereof, to hepcidin). The pH of the incubation buffer is preferably about 6-9.5, more preferably about 6-7. One or more buffers can, for example, be employed to achieve and maintain the desired pH during this step, including borate, phosphate, carbonate, Tris-HCl or Tns-phosphate, acetate, barbital and the like. The particular buffer employed is usually not critical, however, and in a particular assay, one buffer may be preferred over another.

In some embodiments, in relation to the methods of the disclosure, a wash solution may be used to remove uncaptured hepcidins. The wash solution is generally a buffer. The incubation buffers described above are suitable wash solutions. The pH of the wash solution is determined as described above for the incubation buffer. In an embodiment, the pH of the wash solution is about 6-9, more preferably about 6-7. Washes can be done one or more times, preferably, at least three times to reduce the background noise of the assay. The temperature of the wash solution is typically from about 0-40° C., more preferably about 4-30° C. An automated plate washer can be utilized.

Buffers that can be used for said dilution, incubation and/or washing include, for example:

(a) phosphate buffered saline (PBS) containing 0.5% BSA, 0.05% TWEEN20™ detergent (P20), 5 mM EDTA, 0.25% Chaps surfactant, 0.2% beta-gamma globulin, and 0.35M NaCl, pH 7.0;
(b) PBS containing 0.5% BSA and 0.05% P20;
(c) PBS containing 0.5% BSA, 0.05% P20, 5 mM EDTA, and 0.35 M NaCl, pH 6.35;
(d) PBS containing 0.5% BSA, 0.05% P20, 5 mM EDTA, 0.2% beta-gamma globulin, and 0.35 M NaCl;
(e) PBS containing 0.5% BSA, 0.05% P20, 5 mM EDTA, 0.25% Chaps, and 0.35 M NaCl; and
(f) PBS containing 0.5% P20.

Moreover, in some embodiments, the present disclosure concerns a kit that comprises at least one lipocalin-mutein assay of the disclosure. In some further embodiments, the kit may further include various known concentrations of non-control hepcidins. In some still further embodiments, the kits of the disclosure may further comprise a diagnostically acceptable carrier or excipient. In some additional embodiments, the kit may contain one or more instructions for using the kits to diagnose, prognosticate, or monitor one or more diseases or conditions in a subject. In some particular embodiments, the kit may further comprise one or more labels and/or a suitable instrument for signal detection.

In addition, the present disclosure relates to use of the kit for quantitatively determining hepcidin concentration in a biological sample. Furthermore, the present disclosure also features use of the kit for diagnosing a disease or disorder characterized by a non-physiological concentration of hepcidin. In some further embodiments, the kit can also be useful in screening a population of subjects and identifying those subjects who have a disease or disorder characterized by a non-physiological concentration of hepcidin. For example, the disease or disorder can be an anemia, including, but not limited to, anemia resulting from infection, inflammation, chronic disease, and/or cancer.

In yet another aspect, the kit can be used for monitoring the progress of a disease or disorder associated with an altered, e.g. increased or reduced, level of hepcidin concentration. In an additional aspect, the kit can be used for the diagnosis of diseases or disorders associated with an altered, e.g. increased or reduced, level of hepcidin concentration. For example, such diseases or disorder include those involving disturbances of iron metabolism, as well as those diseases involving inflammation, such as chronic inflammatory diseases, including chronic polyarthritis or Crohn's disease, or ulcerative colitis.

In some embodiments, such a disease or disorder may, in some instances, be associated with increased level of hepcidin concentration, e.g. anemia of inflammation, iron-refractory iron deficiency anemia or an anemia associated with chronic kidney disease or cancer or chemotherapy induced anemia.

In contrast, the disease or disorder may, in some other embodiments, be associated with decreased level of hepcidin concentration, such as hereditary hemochromatosis, an iron-loading anemia or Hepatitis C. Hepatitis C, for instance, typically involves a hepatic iron overload, generally via hepcidin synthesis suppression.

In a particular embodiment, the kit can be useful in screening a population of subjects and identifying those subjects who have these diseases or disorders mentioned above.

In this regard, because hepcidin has been shown to be differently affected by inflammation and iron deficiency, one or more kits of the disclosure can be applied to assess iron deficiency in one or more subjects, including subjects with inflammatory conditions.

Pro-inflammatory stimuli contribute to anemia directly by inhibition of erythropoiesis and indirectly by decreasing the iron available for heme synthesis. The latter may be attributed to inflammation-induced increased concentration of the iron regulatory peptide, hepcidin. Elevated hepcidin concentration in turn reduces intestinal iron absorption as well as iron release from macrophages through interaction, internalization, and degradation of the cellular iron exporter ferroportin, resulting in iron sequestration in the reticuloendothelial system. Consequently, the total body iron content is normal, but less iron is released from e.g. macrophages and hepatocytes, and thereby available for erythropoiesis, so there is a functional iron deficiency. The cytokine interleukin 6 (IL-6) is apparently the key inducer of hepcidin synthesis during inflammation (Nemeth et al., J. Clin. Invest. 113, 2004).

In contrast, where hepcidin is affected by iron deficiency, for example, in iron deficiency anemia (IDA), in which there is an absolute iron deficiency, hepcidin is suppressed, which leads to induction of iron absorption from the gut.

In this regard, one or more kits of the disclosed can be used to differentiating absolute iron deficiency from functional iron deficiency (for example, as defined in Kidney Disease: Improving Global Outcomes (KDIGO) Anemia Work Group. KDIGO Clinical Practice Guideline for Anemia in Chronic Kidney Disease. Kidney inter., Suppl. 2012; 2: 279-335). In some embodiments, the diagnosis may initiate the need for further investigations into the cause of the anemia. Overall, the detection of iron deficiency in patients with anemia of inflammation is of meaningful clinical relevance.

In addition, one or more kits of the disclosure can also be used for deciding on a suitable treatment for the stratified patients such as the treatment with one or more modulators of the hepcidin-ferroportin pathway. For example, the treatment with modulators of the hepcidin-ferroportin pathway would not be suitable for patients with iron deficiency anemia (IDA), which is in contrast treatable with e.g. sufficient iron supplementation. In this regard, one or more kits of the disclosure can also be used for for predicting the response to the treatment with one or more modulators of the hepcidin-ferroportin pathway in one or more patients. Such a modulator of the hepcidin-ferroportin pathway, for example, can be an reagent that can neutralize hepcidin expression-stimulating proteins (e.g., bone morphogenetic proteins (BMPs) or cytokines such as IL-6), target the cytokine-signaling pathway (e.g., signal transducer and activator of transcription 3 (STAT3) and bone morphogenetic protein receptors-hemojuvelin-SMAD pathway (BMPRs-HJV-SMADs)), bind and neutralize the hepcidin peptide (e.g., antibodies and other binding molecules), prevent hepcidin binding to ferroportin, interfere with ferroportin-internalization pathway, or inhibit hepcidin expression indirectly by stimulate erythropoiesis (e.g. hypoxia-inducible factor prolyl hydroxylase (HIF-PH) inhibitors) (see, for example, Ganz T, Nemeth E, et al., The hepcidin-ferroportin system as a therapeutic target in anemias and iron overload disorders, Hematology Am Soc Hematol Educ Program. 2011; 2011:538-542).

Furthermore, this diagnosis would prevent unnecessary prescription of iron supplementation where the hepcidin concentration is predominant. For example, one or more kits of the disclosed can be used to predict the response to oral-iron therapy or to intravenous (IV)-iron therapy in one or more patients. For example, where the hepcidin concentration is high, oral-iron therapy would not be so effective since predominant hepcidins would reduce intestinal iron absorption and release of iron from cells in the reticuloendothelial system (e.g. Kupffer cells and splenic macrophages).

In one further embodiment, since anemic patients with low hepcidin concentrations have been observed to show a better response to erythropoietin therapy than patients with high hepcidin concentrations, hepcidin concentrations as measured by the methods or kits of the disclosure can, for instance, be used for predicting the response to ESA (erythropoiesis-stimulating agent) therapy (about 50% of the patients are ESA resistant) for those patients.

A. Lipocalin Muteins of the Disclosure Specifically Binding to Hepcidin

In one aspect, the present disclosure provides one or more lipocalin muteins specifically binding to hepcidin that can be applied in the lipocalin-mutein assays disclosed herein. As used herein, a lipocalin mutein “specifically binds” a target (in the present case, hepcidin), if it is able to discriminate between that target and one or more reference targets, since binding specificity is not an absolute, but a relative property. “Specific binding” can be determined, for example, in accordance with Western blots, ELISA-, RIA-, ECL-, IRMA-tests, FACS, IHC and peptide scans.

In some embodiments, a lipocalin mutein described herein is capable of binding hepcidin, e.g. human hepcidin, including Hepcidin-25, with an affinity measured by a KD of about 10 nM or lower. More preferably, the lipocalin mutein is capable of binding hepcidin, e.g. human hepcidin such as Hepcidin-25 with have an affinity measured by a KD of about 1 nM or lower. The binding affinity of a lipocalin mutein to a selected target (in the present case, hepcidin), can be measured (and thereby KD values of a mutein-target complex be determined) by a multitude of methods known to those skilled in the art. Such methods include, but are not limited to, fluorescence titration, competition ELISA, calorimetric methods, such as isothermal titration calorimetry (ITC), and surface plasmon resonance (BIAcore), as well established in the art.

In some embodiments, a lipocalin mutein described herein is capable of neutralizing the bioactivity of hepcidin, such as Hepcidin-25, preferably with an IC50 value of about 50 nM or lower, for example, as determined by a cell-based assay for Hepcidin-25-induced internalization and degradation of ferroportin.

In some embodiments, a lipocalin mutein described herein may be a human NGAL lipocalin (also “hNGAL”) mutein which has at any two or more amino acids at a position corresponding to position 96, 100, and/or 106 of the linear polypeptide sequence of the mature human NGAL lipocalin a mutated amino acid. The lipocalin mutein further may have one or more such as 2, 3, 4, 5, 6, 7, 8, 9, 10, 11, 12, 13, 14, 15, 16, 17, 18, 19 or even all (i.e. 20) amino acids at a position corresponding to position 36, 40, 41, 49, 52, 68, 70, 72, 73, 77, 79, 81, 96, 100, 103, 106, 125, 127, 132, and/or 134 of the linear polypeptide sequence of mature human NGAL lipocalin (SEQ ID NO: 15) a mutated amino acid. The lipocalin mutein described herein may have in a particularly preferred embodiment at least 75% identity to the sequence of mature human NGAL lipocalin.

In this regard, the lipocalin muteins as well as the methods of generating such lipocalin muteins, as disclosed in WO 2012/022742 (e.g. SEQ ID NOs: 1-14 as contained herein), are hereby incorporated by reference in their entirety. These lipocalin muteins can therefore be applied in the lipocalin-mutein assays described herein.

In some further embodiments, the lipocalin mutein has the amino acid sequence represented by SEQ ID NO: 8 or SEQ ID NO: 10, or a fragment or variant thereof. Preferably, the fragment or variant has a sequence identity or homology of at least a 75%, 80%, 85%, 90% or 95% to the amino acid sequence represented by SEQ ID NO: 8 or SEQ ID NO: 10.

The term “fragment” as used in the present disclosure in connection with the muteins of the disclosure relates to proteins or peptides derived from full-length mature or wild-type lipocalin that are N-terminally and/or C-terminally shortened, i.e. lacking at least one of the N-terminal and/or C-terminal amino acids. Such fragments comprise preferably at least 10, more preferably 20, most preferably 30 or more consecutive amino acids of the primary sequence of mature or wild-type lipocalin and are usually detectable in an immunoassay of mature or wild-type lipocalin.

The term “variant” as used in the present disclosure relates to derivatives of a protein or peptide that comprise modifications of the amino acid sequence, for example by substitution, deletion, insertion or chemical modification. Preferably, such modifications do not reduce the functionality of the protein or peptide. Such variants include proteins, wherein one or more amino acids have been replaced by their respective D-stereoisomers or by amino acids other than the naturally occurring 20 amino acids, such as, for example, ornithine, hydroxyproline, citrulline, homoserine, hydroxylysine, norvaline. However, such substitutions may also be conservative, i.e. an amino acid residue is replaced with a chemically similar amino acid residue. Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan.

The term “human neutrophil gelatinase-associated lipocalin” or “hNGAL” or “lipocalin 2” or “Lcn2” as used herein to refer to the mature human NGAL with the SWISS-PROT/UniProt Data Bank Accession Number P80188 or the mature human NGAL shown in SEQ ID NO: 4. The mature form of this protein has amino acids 21 to 198 of the complete sequence, since a signal peptide of amino acids 1-20 is cleaved off. The protein further has a disulfide bond formed between the amino acid residues at positions 76 and 175 of the mature protein.

B. Exemplary Assays for Carrying Out the Disclosure

### 1. Lipocalin-Mutein Chemical Assay

Principles of conventional immunochemical (IC) assays can generally be used in one or more lipocalin-mutein assays of the disclosure and such a lipocalin-mutein may be called a lipocalin-mutein chemical assay. Examples of IC assays include, but are not limited to, radioimmunoassay (RIA), fluoroluminescence assay (FLA), chemiluminescence assay (CA), and enzyme-linked immunosorbant assay (ELISA). See, for example, Johnstone and Thorpe, Immunochemistry in Practice, Blackwell, 3rd ed., 1996; Current Protocols in Molecular Biology, Ausbul et al. eds., Wiley & Sons, 2003; Immunoassay Methods and Protocols, Ghindilis et al. eds., Blackwell, 2003 as well as U.S. Pat. No. 6,855,508.

In some embodiments, suitable label of the disclosure include those that can be detected directly, such as fluorochrome, chemiluminscent, radioactive labels and those that must be reacted or derivatized to be detected (e.g. by enzymes). Examples of such labels include the radioisotopes P, C, I, H, and J, fluorophores such as rare earth chelates or fluorescein and its derivatives, rhodainine and its derivatives, dansyl, umbelliferone, luceriferases, e g., firefly luciferase and bacterial luciferase (U.S. Pat. No. 4,737,456), luciferin, 2,3-dihydrophthalazinediones, horseradish peroxidase (HRP), alkaline phosphiatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HPP, lactoperoxidase, or microperoxidase, biotin/avidin, biotin/streptavidin, biotin/Streptavidin-β-galactosidase with MUG, spin labels, bacteriophage labels, stable free radicals and the like.

In some preferred embodiments, a fluorimetric or chemilimunescent label may have greater sensitivity in immunoassays compared to a conventional colorimetric label. In an embodiment, the label is HRP.

In a particular embodiment, the label is an enzyme. And in the case of enzyme, the developed color is a direct measurement of the amount of captured tracer molecules (e.g. hepcidin or lipocalin mutein including fragment or variant thereof). For example, when HRP is the label, color may be detected by reacting HRP with a colorimetric substrate and measuring the optical density (O.D.) of the reacted substrate at 450 nm absorbance. Alternatively, HRP may be detected via a fluorogenic substrate by measuring the fluorescence of the reacted substrate with, for example, an Excitation wavelength at 320 nm and/or an Emission wavelength at 430 nm.

### 2. Lipocalin-Mutein Electrochemiluminescent Assay (Lipocalin-Mutein ECLA)

In another aspect, ECLA principles known in the art can be transferrable in the lipocalin-mutein assays of the disclosure, and such a lipocalin-mutein assay may be called lipocalin-mutein ECLA. See, for example, U.S. Pat. Nos. 5,543,112; 5,935,779 and 6,316,607 as well as the patents referenced therein.

In some embodiments of a lipocalin-mutein ECLA, a label of the disclosure may be induced to emit electromagnetic radiation by stimulating the label into an excited state. For example, quantitative measurement of hepcidin concentration in a biological sample may be achieved by comparing the luminescence generated for the sample to a calibration standard curve of luminescences developed with various known concentrations of non-control hepcidins. In an embodiment, the photo-detector measures the light emitted by the label and software for analyzing data collected by the photo-detector is used to calculate the concentration of analyte molecular or ECLA response (in electrochemiluminescence units (ECLU)) of the analyte molecule.

In a particular embodiment, the label is a metal chelate that luminesces under the electrochemical conditions imposed by a lipocalin-mutein ECLA. The metal can be, for example, a transition metal (such as a d-block transition metal) or a rare earth metal. In an embodiment, the metal is ruthenium, osmium, rhenium, iridium, rhodium, platinum, indium, palladium, molybdenum, technetium, copper, chromium, or tungsten. In an embodiment, the metal is ruthenium or osmium.

In some further embodiments, one or more ligands can be linked to the metal chelate, which ligands are usually heterocyclic or organic in nature, and play a role in determining whether the metal chelate is soluble in an aqueous environment or in an organic or other nonaqueous environment. The ligands can, for example, be polydentate, and can be substituted. Polydentate ligands include aromatic and aliphatic ligands. Suitable aromatic polydentate ligands include aromatic heterocyclic ligands. In an embodiment, the aromatic heterocyclic ligands are nitrogen-containing, such as, for example, bipyridyl, bipyrazyl, terpyridyl, and phenanthrolyl. Suitable substituents include for example, alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus containing groups, and the carboxylate ester of N-hydroxysuccinimide. The chelate can have one or more monodentate ligands, a wide variety of which are known to the art. Suitable monodentate ligands include, for example, carbon monoxide, cyanides, isocyanides, halides, and aliphatic, aromatic and heterocyclic phosphines, amines, stilbenes and arsines.

In some embodiments, examples of chelates suitable for being used as lipocalin-mutein ECLA labels as disclosed herein are bis [(4,4′-carbomethoxy)-2,2′-bipyridine] 2-[3-(4-methyl-2,2′-bipyridine-4-yl)propyl]-1,3-dioxolane ruthenium (II); bis(2,2′bipyridine) [4-(butan-1-al)-4′-methyl-2,2′-bipyridine]ruthenium (II); bis(2,2′-bipyridine) [4-(4′methyl-2,2′-bipyridine-4′-yl)-butyric acid]ruthenium (II); tris(2,2′bipyridine) ruthenium (II); (2,2′-bipyridine) [bis-bis(1,2-diphenylphosphino)ethylene] 2-[3-(4-methyl-2,2′-bipyridine-4′-yl)propyl]-1,3-dioxolane osmium (II); bis(2,2′-bipyridine) [4-(4′-methyl-2,2′-bipyridine)-butylamine]ruthenium (II); bis (2,2 ‘-bipyridine) [I-bromo-4(4’-methy 1-2,2′-bipyridine-4-yl)butane]ruthenium (II); bis(2,2′-bipyridine) maleimidohexanoic acid, 4-methyl-2,2′-bipyridine-4′-butylamide ruthenium (II). Additional labels suitable for ECLA are described in U.S. Pat. Nos. 5,591,581; 6,271,041; 6,316,607; and 6,451,225. In an embodiment, the label moiety can be Ru(bpy)₃²⁺ or ORI-TAG™ NHS ester (IGEN International Inc., Gaithersburg, Mass.).

In some embodiments, the label utilized is one that effectively results in the emission of a detectable, and if desired, quantifiable, emission of electromagnetic energy.

In a particular embodiment, the label suitable for a lipocalin-mutein ECLA of the disclosure, is a SULFO-TAG-conjugated streptavidin (e.g. supplied by Meso Scale Discovery).

The following non-limiting Examples and Figures further illustrate various aspects of the present disclosure.

### VI. EXAMPLES

### Example 1

### Characterization of Exemplary Lipocalin Muteins Used to Set Up Lipocalin-Mutein Assays of the Disclosure

With the goal to develop highly sensitive lipocalin-mutein assays that are not only with a low limit of detection and suitable for high-throughput analyses (e.g. quantitative measurement) of hepcidin concentrations in different biological samples (e.g. body fluids) but also economical and practical for being widely used in clinical settings, the inventors developed several assay formats using two hepcidin-specific lipocalin muteins (SEQ ID NO: 10 and SEQ ID NO: 8).

The selection, identification, production and characterization of hepcidin-specific lipocalin muteins are described in WO 2012/022742. The binding affinity of the lipocalin muteins of the SEQ ID NO: 10 and the SEQ ID NO: 8 to non-modified Hepcidin-25 in solution was evaluated in a competition ELISA approach as described in Example 7 of WO 2012/022742. In addition, as described in Example 9 of WO 2012/022742, an in vitro cell-based assay, based on hepcidin-induced internalization and degradation of its receptor, ferroportin, was implemented to measure the neutralization activity of the lipocalin muteins. IC50 values of the two hepcidin-specific lipocalin muteins (which are SEQ ID NO: 10 and SEQ ID NO: 8, respectively, as disclosed in WO2012/022742) as measured in said experiments are reproduced in Table 1.

TABLE 1

| | SEQ ID NO: 8 | SEQ ID NO: 10 |
|---|---|---|
| Assay | IC50 [nM] | IC50 [nM] |
| Competitive Binding Assay | 0.18 | 0.1 |
| In Vitro Neutralization Activity | 25 | 31 |
| Assay | | |

As shown in Table 1, the cell-based internalization assay demonstrated the ability of the lipocalin muteins to inhibit hepcidin-induced internalization of ferroportin in vitro. The high concentration of Hepcidin-25 (e.g. 40 nM) used in the cell-based assay for optimal induction of ferroportin-GFP (green fluorescent protein) internalization limits the sensitivity of such assay and explains the high IC50 values, when compared with the lipocalin muteins' pM-binding affinity for Hepcidin-25 in the competition ELISA approach where a 25 pM concentration of Hepcidin-25 was used.

### Example 2

### Preparation of MSD Sulfo-Tag Conjugation to Label Control Hepcidin or to Label Lipocalin Muteins

The inventors exemplarily used a MSD (MesoScale Discovery) Sulfo-Tag, namely the NHS Ester (MSD, Cat. No: R91AN-2), which is an amine-reactive, N-hydroxysuccinimide ester and may be coupled to primary amine acid groups of proteins and peptides (e.g. lysine side chains, protein N-terminus) under mild basic conditions to form a stable bond. The MSD Sulfo-Tag conjugation was generated according to a protocol provided by MSD (version 1.1, 2006).

A purified solution of the peptide (e.g., control hepcidin) or the protein (e.g., lipocalin muteins) was prepared in preservative-free PBS (Phosphate Buffered Saline) with a pH of 7.4. The Sulfo-Tag was reconstituted immediately prior to use with cold, distilled water to generate a stock solution of 10 nmol/μl. A calculated volume of reconstituted Sulfo-Tag was added to the solution in order to reach a molar ratio of 6:1 (Sulfo-Tag: peptide/protein) and incubated at room temperature (“RT”) for 2 h. The reaction was shielded from light. The reaction efficiency was increased by shaking of the solution. The Sulfo-Tag labeled protein or peptide was separated from unconjugated Sulfo-Tag by purification via a ZEBA Desalt Spin Column (Thermo Scientific, Cat.No. 89889). The success of the labeling was calculated based on the colorimetric measured protein concentration (e.g., Bradford, BioRad) while the concentration of Sulfo-Tag label in the conjugation form was measured via absorbance of such Tag at 455 nm. An optimal molar ratio of between 2:1 and 10:1 (Sulfo-Tag: peptide/protein) was achieved. The labeled protein or peptide was aliquoted and stored at −20° C. after testing its biological activity.

### Example 3

### Quantification of Hepcidin in Human Serum Through a Competition Assay Relying on Electrochemiluminescense Detection (ECLA) of Sulfo-Tag Labeled Control Hepcidin

The inventors set up this assay format based on the binding competition between unlabeled hepcidins (non-control hepcidins) and Sulfo-Tag-labeled control hepcidins (made according to Example 2) to lipocalin muteins of SEQ ID NO: 10. The hepcidin concentrations in two different human serum samples were determined via a quantitative ECLA approach.

A 384-well MSD plate (MesoScale Discovery, Cat. No: L25XA) was coated with 20 μL of lipocalin muteins of SEQ ID NO: 10 at a concentration of 5 μg/mL in PBS over night at 4° C. After washing the coated wells with PBS/0.05% Tween20, the wells were blocked with for 1 h at room temperature 60 μL blocking buffer, e.g. 2% BSA (Bovine Serum Albumin, Roth, Cat. No: 8076.3) in PBS/0.1% Tween20.

A fixed concentration of about 0.6 nM Sulfo-Tag labeled control hepcidins were incubated in solution with (i) various known concentrations of non-control hepcidins (PeptaNova, Cat. No: 4392-s) in PBS/0.1% Tween20/2% BSA (concentrations starting from 5 μg/mL, 1:3 serially diluted via 12 points) for the generation of a standard curve and with (ii) two human serum samples for the determination of their hepcidin content, respectively (e.g. in different wells). After 20 min. of incubation at room temperature, 20 μL of the reaction mixture was transferred to the lipocalin-mutein-coated MSD plate to capture hepcidins (Sulfo-Tag labeled as well as unlabeled) and incubated for 20 min. at room temperature. Afterwards, 60 μL MSD Read Buffer T (4×) with Surfactant (2× final concentration diluted in distilled water, MesoScale Discovery, Cat. No: R92TC) was added to each well and the plate was read within 15 min to measure the bound hepcidin-C-Bio-Sulfo-Tag. All incubation steps were performed with shaking at 300 rpm (revolutions per minute). The plate was washed 5× with 80 μL PBS/0.05% Tween20 between the different steps, using a Biotek (ELx405 select CW) washer.

The Sulfo-tag emits light when oxidized at an electrode in an appropriate chemical environment according to Meso Scale Discovery (MSD) Technology. The generated ECL signals were detected using the SECTOR Imager 2400 (MesoScale Discovery). The evaluation was performed as follows: ECL signals were plotted versus various known hepcidin concentrations to generate standard curves. The standard curves were fitted by nonlinear regression with the 4 Parameter Logistic model (all parameters variable) using GraphPad Prism 4 software.

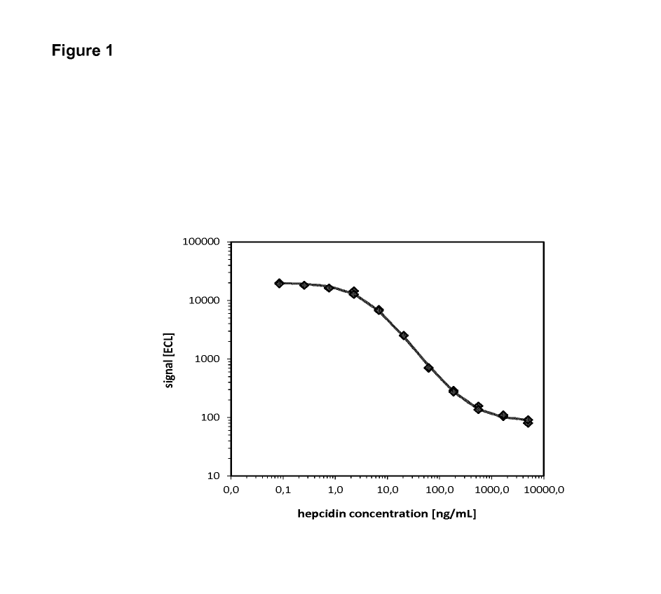

An exemplary standard curve with at least 80%-120% recovery of human hepcidin was generated and is shown in FIG. 1, which demonstrates a linear range from 1 ng/mL up to 185 ng/mL. In this regard, the decreased levels of electrochemiluminescenses (ECL signals) generated by control hepcidins (the tracer molecules) via Sulfo-Tag were a direct reflection of the various concentrations of non-control hepcidins that competed with control hepcidins for binding to the immobilized lipocalin muteins.

By the same token, the ECL signal level generated via Sulfo-Tag by control hepcidins that were captured on the plate and competed for binding with the non-control hepcidins in one of the two human serum samples, was plotted on the standard curve, and thereby was correlated to such serum sample's hepcidin concentration. Similarly, the other human serum sample's hepcidin concentration was measured based on the same principle. (Data is summarized in Example 6).

### Example 4

### Quantification of Hepcidin in Human Serum Using an Enzyme-Linked Competition Assay Based on Fluorescence Detection of HRP

The inventors set up this assay format based on the binding competition between unconjugated hepcidins (non-control hepcidins) and biotinylated control hepcidins (hepcidin-C-Bios) to lipocalin muteins of SEQ ID NO: 10, which were directly coated on a microplate. The hepcidin concentrations in two different human serum samples were determined via a quantitative enzyme-linked fluorescence-based assay.

A 384-well plate (Greiner Bio-One, Cat. No. 781077) was coated with 20 μL of lipocalin muteins of SEQ ID NO: 10 at a concentration of 5 μg/mL in PBS over night at 4° C. After washing the coated wells with PBS/0.05% Tween20, the wells were blocked with 100 μL blocking buffer (2% BSA in PBS/0.1% Tween20) for 1 h at room temperature.

A fixed concentration of 0.6 nM C-terminal biotinylated control hepcidins (hepcidin-C-Bios, Bachem AG) was incubated in solution with either (i) various known concentrations of non-control hepcidins (Peptallova, Cat.No. 4392-s) in PBS/0.1% Tween20/2% BSA (concentrations starting from 5 μg/mL, 1:3 serially diluted via 12 points) for the generation of a standard curve and with (ii) human serum samples for the determination of their hepcidin content, respectively (e.g. in different wells). After 20 min. of incubation at room temperature, 20 μL of the reaction mixture was transferred to the lipocalin-mutein-coated plate.

After 20 min. of incubation at RT, the supernatants were discarded. The amount of hepcidin-C-Bios bound on the plate was detected via Extravidin-horseradish peroxidase (HRP) using QuantaBlue as a substrate for HRP. Therefore, 20 μL Extravidin-HRPs (Sigma Aldrich, Cat.No.E2886) were added at a dilution of 1:5000 in PBS/0.1% Tween20/2% BSA and incubated for 1 h at RT. Afterwards, 20 μL of QuantaBlu Fluorogenic Substrate (1:10 dilution of QuantaBlu Stable Peroxide Solution in QuantaBlu Substrate Solution, Pierce, Cat. No. 15162) was added to each well. The plate was read after 20-30 min., using a GENios Plus microplate reader (Tecan Group Ltd.) with an Excitation wavelength at 320 nm and an Emission wavelength at 430 nm to detect the relative fluorescence units (RFU) generated by HRP.

All incubation steps were performed with shaking at 300 rpm. The plate was washed 5× with 80 μL PBS/0.05% Tween20 using a Biotek ELx405 select CW washer in between the different steps.

The evaluation was performed as follows: relative fluorescence units (RFUs) were plotted versus various known hepcidin concentrations to generate standard curves. The standard curves were fitted by nonlinear regression with the 4 Parameter Logistic model (all parameters variable) using GraphPad Prism 4 software.

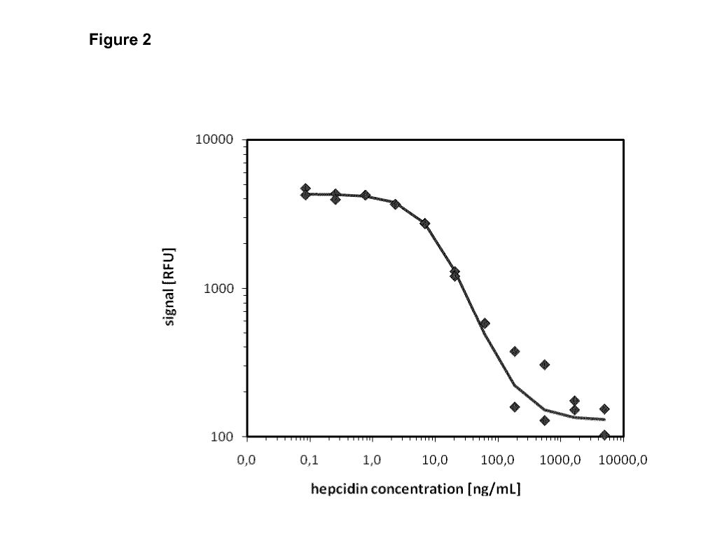

An exemplary standard curve with at least 80%-120% recovery of human hepcidin was generated and is shown in FIG. 2, which demonstrates a linear range from 2 ng/mL up to 185 ng/mL. In this regard, the decreased levels of relative fluorescence units (RFUs) generated by hepcidin-C-Bios (the tracer molecules) via Extravidin-HRP were a direct reflection of the various concentration of non-control hepcidins that competed with hepcidin-C-Bios for binding to the immobilized lipocalin muteins.

By the same token, the RFU generated via Extravidin-HRP by hepcidin-C-Bios that were captured on the plate and competed for binding with non-control hepcidins in one of the two human serum samples, was plotted on the standard curve, and thereby was correlated to such serum sample's hepcidin concentrations. Similarly, the other human serum sample's hepcidin concentration was measured based on the same principle. (Data is summarized in Example 6).

### Example 5

### Comparing the Two Lipocalin-Mutein Assays Described in Example 3 and Example 4

A comparison of the exemplary standard curves of the two competition assays described in Examples 3 and Example 4 revealed a comparable linear range with at least 80-120% recovery for the determination of hepcidin concentrations (Table 2). As shown by the exemplary standard curves in FIGS. 1 and 2, the lowest hepcidin concentrations detected were between 1-2 ng/ml while the highest concentrations detected were 185 ng/ml in both assays. Thus, both assays would be well suited for high-throughput analysis of hepcidin concentrations in different biological samples.

TABLE 2

| | linear range |
|---|---|
| Assay | (Hepcidin) |
| Enzyme-linked fluorescence -based assay in Example 4 | 2¹-185 ng/ml |
| ECLA in Example 3 | 1¹-185 ng/ml |

### Example 6

### Comparing the Results Measured by the Two Lipocalin-Mutein Assays Described in Example 3 and Example 4 with the Results Measured by a Mass Spectrometry (MS) Method

While the hepcidin concentrations of two different human serum samples were measured, using the electrochemiluminescence-based assay (ECLA) described in Example 3 and the enzyme-linked fluorescence-based assay described in Example 4, respectively, such human serum samples' hepcidin concentrations had been measured in a liquid chromatography tandem mass spectrometry (MS) approach (as essentially descried in Murphy A T, Witcher D R, Luan P, Wroblewski V J; Blood. 2007 Aug. 1; 110(3):1048-54. Epub 2007 Apr. 13) by Lilly Research Laboratories at Lilly Corporate Center in Indianapolis (US). Table 3 below summarizes and compares the results obtained by the three different assays.

TABLE 3

| | Method | | | | |
|---|---|---|---|---|---|
| | Example 4 | | Example 3 | | |
| | mean | | mean | | |
| | hepcidin | | hepcidin | | MS ap- |
| | conc. of 2 | | conc. of 2 | | proach |
| Human | measure- | N = 2 | measure- | N = 2 | hepcidin |
| serum | ments | SD conc. | ments | SD conc. | conc. |
| samples | [ng/mL] | [ng/mL] | [ng/mL] | [ng/mL] | [ng/mL] |
| #1 | 18.98 | 2.64 | 13.24 | 0.99 | 11.12 |
| #2 | 73.21 | 2.72 | 67.72 | 7.75 | 72.49 |

As shown in Table 3, both lipocalin-mutein assays accurately determined the hepcidin concentrations within the same range as expected from the MS-based approach. Thus, both assays can be used for high-throughput analyses of hepcidin in different biological samples with an accuracy comparable with the MS-approach but at lower cost.

### Example 7

### An Alternative Competition Assay Based on Electrochemiluminescense Detection (ECLA) of IgG-Sulfo-Tag

The inventors set up this assay format based on the binding competition between Neutravidin captured, C-terminal biotinylated control hepcidins (hepcidin-C-Bios) and unconjugated hepcidins (non-control hepcidins) to lipocalin muteins of SEQ ID NO: 8.

A 96-well MSD plate (MesoScale Discovery, Cat. No. L15XA) was coated with 25 μL of Neutravidins (Thermo Scientific, Cat. No. 31000) at a concentration of 5 μg/mL in PBS over night at 4° C. After washing the Neutravidin-coated wells with PBS/0.05% Tween20, the wells were blocked with 150 μL blocking buffer (1% Casein (Sigma Aldrich, Cat. No. C7078) in PBS/0.1% Tween20) for 1 h at room temperature. Afterwards, 25 μL of 1 μg/mL human hepcidin-C-Bio (Bachem AG, custom synthesized) in PBS/0.1% Tween20 was added to be captured on the plate.

A constant concentration of 0.5 nM lipocalin muteins of SEQ ID NO: 8, as tracer molecules, were incubated in solution with various known concentrations of non-control hepcidins (Peptallova, Cat. No. 4392-s) in PBS/0.1% Tween20/2% BSA (concentrations starting from 5 μg/mL, 1:2 serially diluted via 15 points) for the generation of a standard curve. After 1 h of pre-incubation at room temperature, 25 μL of the reaction mixture was transferred to the MSD plate, allowing the free lipocalin muteins to bind to hepcidin-C-Bios captured on the microplate within 1 h at RT.

The amount of lipocalin muteins bound on the plate was detected by the addition of 25 μL mixture of rabbit anti-NGAL polyclonal primary IgGs (1 μg/mL; custom-produced at BioGenes, Cat. No. PL713) and polyclonal goat anti-rabbit IgG Sulfo-Tag labeled antibodies (1 μg/mL; MesoScale Discovery, Cat. No. R32AB), followed by incubation for 1 h at RT. Finally, 150 μL MSD Read Buffer T (4×) with Surfactant (2× final concentration diluted in distilled water, MesoScale Discovery, Cat. No. R92TC) was added to each well and the plate was read within 15 min.

All incubation steps were performed with shaking at 300 rpm. The plate was washed 5× with 300 μL PBS/0.05% Tween20 using a Biotek ELx50 washer in between the different steps.

The Sulfo-tag emits light when oxidized at an electrode in an appropriate chemical environment according to Meso Scale Discovery (MSD) Technology. The generated ECL signals were measured using the SECTOR Imager 2400 (MesoScale Discovery). The evaluation was performed as follows: ECL signals were plotted versus various known hepcidin concentrations. The standard curves were fitted by nonlinear regression with the 4 Parameter Logistic model (all parameters variable) using GraphPad Prism 4 software.

In an exemplary standard curve generated therefrom with at least 80%-120% recovery of lipocalin mutein, the linear range was from 2 ng/mL up to 1250 ng/mL (data not shown). In this regard, the decreased levels of electrochemiluminescenses (ECL signals) generated by lipocalin-muteins (the tracer molecules) via Sulfo-Tag were a direct reflection of the amount of non-control hepcidins that competed for binding to the lipocalin-muteins with hepcidin-C-Bios captured on the microplate.

### Example 8

### An Alternative Competition Assay Based on Electrochemiluminescense Detection (ECLA) of Sulfo-Tag Labeled Lipocalin Mutein

The inventors set up this assay format based on the binding competition between C-terminal biotinylated control hepcidins (hepcidin-C-Bios) and unconjugated hepcidins (non-control hepcidins) to lipocalin-muteins of SEQ ID NO: 8.

A 96-well MSD plate (MesoScale Discovery, Cat. No. L15XA) was coated with 25 μL of Neutravidins (Thermo Scientific, Cat. No. 31000) at a concentration of 5 μg/mL in PBS (Phosphate Buffered Saline) over night at 4° C. After washing the Neutravidin-coated wells with PBS/0.05% Tween20, the wells were blocked with 150 μL blocking buffer (3% BSA (Bovine Serum Albumin, Roth, Cat. No. 8076.3) in PBS/0.1% Tween20) for 1 h at room temperature and 25 μL of 1 μg/mL human hepcidin-C-Bios (Bachem AG, custom synthesized) in PBS/0.1% Tween20 was added to be captured on the plate.

A fixed concentration of about 0.5 nM lipocalin muteins of SEQ ID NO: 8, which had been labeled with Sulfo-Tag (NHS Ester, MSD, Cat. No: R91AN-2) as illustrated in Example 2, was incubated in solution with various known concentrations of non-control hepcidins (Peptallova, Cat. No. 4392-s) in PBS/0.1% Tween20/2% BSA (concentrations starting from 5 μg/mL, 1:2 serially diluted via 24 points) for the generation of a standard curve. After 1 h of pre-incubation at room temperature, 25 μL of the reaction mixture was transferred to the MSD plate, allowing the free lipocalin muteins to bind hepcidin-C-Bios captured on the microplate within 1 h at RT. Afterwards, 150 μL MSD Read Buffer T (4×) with Surfactant (2× final concentration diluted in distilled water, MesoScale Discovery, Cat. No. R92TC) was added to each well and the plate was read within 15 min.

All incubation steps were performed with shaking at 300 rpm. The plate was washed 5× with 300 μL PBS/0.05% Tween20 using a Biotek ELx50 washer in between the different steps.

The Sulfo-tag emits light when oxidized at an electrode in an appropriate chemical environment according to Meso Scale Discovery (MSD) Technology. The generated ECL signals were measured using the SECTOR Imager 2400 (MesoScale Discovery). The evaluation was performed as follows: ECL signals were plotted versus various known hepcidin concentrations. The standard curves were fitted by nonlinear regression with the 4 Parameter Logistic model (all parameters variable) using GraphPad Prism 4 software.

In an exemplary standard curve with at least 80%-120% recovery of lipocalin mutein, the linear range was from 80 ng/mL up to 5000 ng/mL (data not shown). In this regard, the decreased levels of electrochemiluminescenses (ECL signals) generated by lipocalin-muteins (the tracer molecules) via Sulfo-Tag were a direct reflection of the amount of non-control hepcidins that competed for binding to lipocalin-muteins with the hepcidin-C-Bios captured on the plate.

### Example 9

### An Alternative Competition Assay Based on Electrochemiluminescense Detection (ECLA) of Streptavidin-Sulfo-Tag

The inventors set up this assay format based on the binding competition between C-terminal biotinylated control hepcidins (hepcidin-C-Bios) and unconjugated hepcidins (non-control hepcidins) to lipocalin muteins of SEQ ID NO: 8 that were directly coated on a microplate.

A 96-well MSD plate (MesoScale Discovery, Cat. No. L15XA) was coated with 25 μL of lipocalin muteins of SEQ ID NO: 8 at a concentration of 5 μg/mL in PBS over night at 4° C. After washing the lipocalin-mutein coated wells with PBS/0.05% Tween20, the wells were blocked with 150 μL blocking buffer (1% Casein (Sigma Aldrich, Cat. No. C7078) in PBS/0.1% Tween20) for 1 h at room temperature.

A fixed concentration of 0.5 nM C-terminal biotinylated control hepcidins (hepcidin-C-Bios, Bachem AG, custom synthesized) was incubated in solution with various known concentrations of non-control hepcidins (Peptallova, Cat. No. 4392-s) in PBS/0.1% Tween20/2% BSA (concentrations starting from 5 μg/mL, 1:2 serially diluted via 15 points) for the generation of a standard curve.

After 5 min. of incubation at room temperature, 25 μL of the reaction mixture was transferred to the lipocalin-mutein-coated MSD plate for further 20 min. at RT. To determine the amount of captured hepcidin-C-Bios, 25 μL of 1 μg/mL Streptavidin-Sulfo-Tag (MesoScale Discovery, Cat. No. R32AD) in PBS/0.5% BSA/0.5% Tween20 was added for 1 h at RT. Afterwards, 150 μL MSD Read Buffer T (4×) with Surfactant (2× final concentration diluted in distilled water, MesoScale Discovery, Cat. No. R92TC) was added to each well and the plate was read within 15 min.

All incubation steps were performed with shaking at 300 rpm. The plate was washed 5× with 300 μL PBS/0.05% Tween20 using a Biotek ELx50 washer in between the different steps.

The Sulfo-tag emits light when oxidized at an electrode in an appropriate chemical environment according to Meso Scale Discovery (MSD) Technology. The generated ECL signals were measured using the SECTOR Imager 2400 (MesoScale Discovery). The evaluation was performed as follows: ECL signals were plotted versus various known hepcidin concentrations. The standard curves were fitted by nonlinear regression with the 4 Parameter Logistic model (all parameters variable) using GraphPad Prism 4 software.

In an exemplary standard curve with at least 80%-120% recovery of human hepcidin, the linear range was from 20 ng/mL up to 5000 ng/mL (data not shown). In this regard, the decreased levels of electrochemiluminescenses (ECL signals) generated by with hepcidin-C-Bios (the tracer molecules) via Sulfo-Tag were a direct reflection of the amount of non-control hepcidins that competed with hepcidin-C-Bios for binding to the immobilized lipocalin muteins.

### Example 10

### An Alternative Enzyme-Linked Competition Binding Assay Based on Absorption at 450 nm of HRP

The inventors set up this assay format based on the binding competition between unconjugated hepcidins (non-control hepcidins) and biotinylated control hepcidins (hepcidin-C-Bios) to lipocalin muteins of SEQ ID NO: 10 that were directly coated on a microplate.

A 96-well plate (Greiner Bio-One, Cat.No. 655061) was coated with 100 μL of lipocalin muteins of SEQ ID NO: 10 at a concentration of 5 μg/mL in PBS overnight at 4° C. After washing the lipocalin-mutein-coated wells with PBS/0.05% Tween20, the wells were blocked with 300 μL blocking buffer (2% BSA in PBS/0.1% Tween) for 1 h at room temperature.

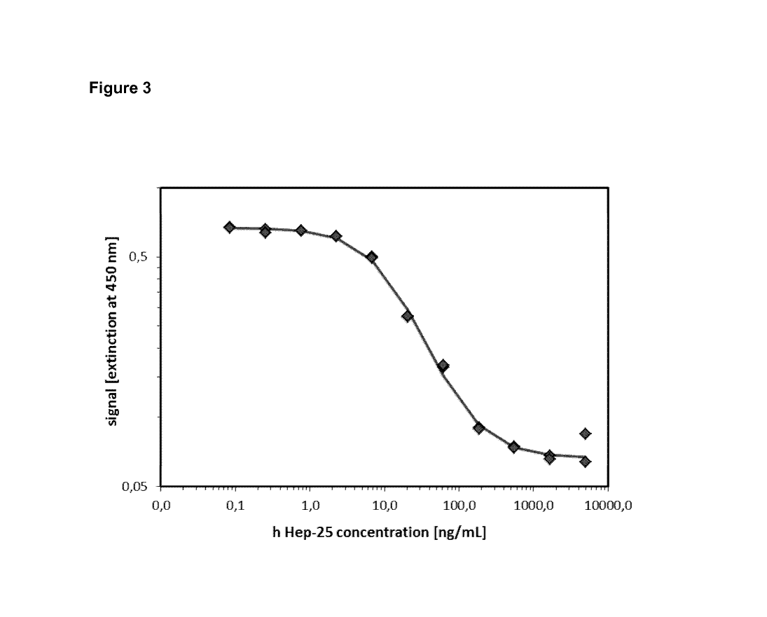

A fixed concentration of 0.3 nM C-terminal biotinylated hepcidins (hepcidin-C-Bio, Bachem AG) was incubated in solution for 20 mins. with various known concentrations of non-control hepcidins (Peptallova, Cat.No. 4392-s) in PBS/0.1% Tween20/2% BSA (concentrations starting from 5 μg/mL, 1:3 serially diluted via 12 points) for the generation of a standard curve as shown in FIG. 3. After 20 min. of incubation at room temperature, 20 μL of the reaction mixture was transferred to the lipocalin-mutein-coated plate.

After 1 h of incubation at RT, the supernatants were discarded. The hepcidin-C-Bios bound on the plate were detected via Extravidin-HRP using TMB (3,3′,5,5′-Tetramethylbenzidine) as a substrate. Therefore, 100 μL Extravidin-HRPs (Sigma Aldrich, Cat.No.E2886) was added at a dilution of 1:5000 in PBS/0.1% Tween20/2% BSA and was incubated for 30 min. at RT. After incubation at RT for 30 min., the supernatants were discarded. 100 μL of 1-Step™ Ultra TMB-ELISA liquid substrate (3,3′,5,5′-Tetramethylbenzidine; undiluted, Thermo Scientific, Cat. No. 34028) was added and was incubated for 20 min. Afterwards, 100 μl of Stop Solution (0.5 M H₂SO₄, Roth, Cat. No. X876.1) was added to each well and the plate was measured within 10 min. at 450 nm using a Safire microplate reader (Tecan Group Ltd.) to determine the extinction values.

All incubation steps were performed with shaking at 300 rpm. The plate was manually washed 4× with 300 μL PBS/0.05% Tween20 in between the different steps.

The evaluation was performed as follows: extinction values at 450 nm were plotted versus various known hepcidin concentrations. The standard curves were fitted by nonlinear regression with the 4 Parameter Logistic model (all parameters variable) using GraphPad Prism 4 software.

A standard curve with at least 80%-120% recovery of human hepcidin was generated and is exemplary shown in FIG. 3, which demonstrates a linear range from 0.8 ng/mL up to 555 ng/mL. In this regard, the decreased levels of extinction values generated by hepcidin-C-Bios (the tracer molecules) via HRP were a direct reflection of the amount of non-control hepcidins that competed with hepcidin-C-Bios for binding to the immobilized lipocalin muteins.

Embodiments illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art, and that such modifications and variations are considered to be within the scope of this invention. All patents, patent applications, textbooks and peer-reviewed publications described herein are hereby incorporated by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. Each of the narrower species and sub-generic groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group. Further embodiments will become apparent from the following claims.

1-36. (canceled)

37. A lipocalin-mutein assay, which comprises:
(i) a phase,
(ii) a lipocalin mutein or fragment or variant thereof that specifically binds to hepcidin, and
(iii) a control hepcidin or fragment or variant thereof;
wherein the control hepcidin or fragment or variant thereof competes with a non-control hepcidin in a biological sample for binding to the lipocalin mutein or fragment or variant thereof, when such sample is contacted with the assay; wherein either (ii) or (iii) serves as a tracer molecule that is captured on the phase by a capture reagent, which is the other one of (ii) or (iii); and wherein the tracer molecule can be detected and/or quantified via a label.

38. The lipocalin-mutein assay of claim 37, wherein the tracer molecule is at the concentration range of about 0.1 nM-3 nM.

39. The lipocalin-mutein assay of claim 37, which assay comprises a binding agent, wherein the control hepcidin or fragment or variant thereof is conjugated to a moiety and thereby can be captured by such binding agent.

40. The lipocalin-mutein assay of claim 39, which assay comprises a blocking agent.

41. The lipocalin-mutein assay of claim 37, wherein the mean value of the concentration of non-control hepcidins in a biological sample as measured by the lipocalin-mutein assay is within the same range of the mean value of the concentration of non-control hepcidins in a corresponding sample as measured by a mass spectrometry (MS) assay, which MS assay is essentially described in Murphy A T et al. Blood. 2007; 110:1048-1054.

42. The lipocalin-mutein assay of claim 37, wherein the lipocalin mutein or fragment or variant thereof is capable of binding hepcidin with an affinity measured by a KD of about 10 nM or lower.

43. The lipocalin-mutein assay of claim 37, wherein the lipocalin mutein is a hNGAL mutein, wherein the hNGAL mutein has at any two or more amino acids at a position corresponding to position 96, 100, and/or 106 of the linear polypeptide sequence of the mature hNGAL lipocalin a mutated amino acid, and wherein the hNGAL mutein further has one or more amino acids at a position corresponding to position 36, 40, 41, 49, 52, 68, 70, 72, 73, 77, 79, 81, 103, 125, 127, 132, and/or 134 of the linear polypeptide sequence of mature hNGAL lipocalin (SEQ ID NO: 15) a mutated amino acid.

44. The lipocalin-mutein assay of claim 43, wherein the hNGAL mutein has the amino acid sequence represented by SEQ ID NO: 8 or SEQ ID NO: 10, or a fragment or variant thereof.

45. A method of preparing a lipocalin-mutein assay, comprising the steps of:
(i) immobilizing one or more lipocalin muteins or fragments or variants thereof on a phase, which lipocalin muteins or fragments or variants hereof specifically bind to hepcidin; and
(ii) providing one or more control hepcidins or fragments or variants at the concentration range of 0.1 nM-3 nM.

46. The method of claim 45, further comprising the step of adding a blocking agent after step (i).

47. A method of preparing a lipocalin-mutein assay, comprising the steps of:
(i) immobilizing one or more binding agents on a phase;
(ii) providing one or more control hepcidins or fragments or variants thereof, which control hepcidins or fragments or variants thereof are conjugated to a moiety and thereby can be captured by the binding agents; and
(iii) providing lipocalin muteins or fragments or variants thereof at the concentration range of 0.1 nM-3 nM, which lipocalin muteins or fragments or variants hereof specifically bind to hepcidin.

48. The method of claim 47, further comprising the step of adding a blocking agent after step (i).

49. A method for quantitatively measuring a biological sample's hepcidin concentration, comprising the steps of:
(i) contacting a biological sample obtained from a subject with the lipocalin-mutein assay of claim 37;
(ii) measuring the signal level generated by one or more tracer molecules, captured on the phase, via one or more labels and/or a suitable instrument for signal detection; and
(iii) correlating the signal level on a standard curve with the biological sample's hepcidin concentration.

50. The method of claim 49, further comprising the steps of:
(iv) contacting various known concentrations of non-control hepcidins with the lipocalin-mutein assay; and
(v) measuring the signal levels corresponding to the various concentrations of step (iv) to generate a standard curve, which signal levels are generated by one or more tracer molecules, captured on the phase, via one or more labels and/or a suitable instrument for signal detection.

51. The method of claim 50, wherein the steps (iv) and (v) are carried out simultaneously with steps (i) and (ii), respectively.

52. A method for identifying an altered level of hepcidin concentration in a subject, comprising:
(i) quantitatively measuring a biological sample's hepcidin concentration using a method according to claim 49, wherein the biological sample is obtained from the subject; and
(ii) comparing the hepcidin concentration measured in step (i) with the prior-measured hepcidin concentration(s) of one or more corresponding sample(s) obtained from the subject.

53. A method for identifying an altered level of hepcidin concentration in a subject, comprising:
(i) quantitatively measuring a biological sample's hepcidin concentration using the method according to claim 49, wherein the biological sample is obtained from the subject; and
(ii) quantitatively measuring hepcidin concentration(s) of one or more corresponding sample(s) using the method according to claim 49, wherein the corresponding sample(s) are obtained from the subject; and
(iii) comparing the hepcidin concentration measured in step (i) with the hepcidin concentration(s) of the one or more corresponding sample(s) measured in step (ii).

54. A method for diagnosing a disease or disorder characterized by a non-physiological concentration of hepcidin in a subject, comprising:
(i) quantitatively determining a biological sample's hepcidin concentration according to claim 49, wherein the biological sample is obtained from the subject; and
(ii) analyzing whether the hepcidin concentration measured in step (i) is non-physiological,
wherein the non-physiological concentration of hepcidin is an indicative of the disease or disorder in the subject.

55. The method of claim 54, wherein the analysis in step (ii) including comparing the hepcidin concentration measured in step (i) with the hepcidin concentration of a control sample, which is known to possess a normal hepcidin concentration.
